(19) United States
(12) Patent Application Publication
(10) Pub. No.: US 2009/0248162 A1
(43) Pub. Date: Oct. 1, 2009

(54) Microparticle delivery syringe and needle for placing suspensions and removing vehicle fluid

(75) Inventor: Steven Marquis Peckham, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/055,033

(22) Filed: Mar. 25, 2008

(51) Int. Cl. A61F 2/44

(52) U.S. Cl. 623/17.16

(57) ABSTRACT

Described are reinforced, load-bearing medical implants and methods for preparing and using them. In one aspect a load-bearing orthopedic implant includes a reinforcing polymeric structure including a continuous piece having an internal interconnected porous network. A hardened, calcium-containing material is formed as a non-sintered load-bearing body encompassing the reinforcing structure and filling the internal porous network of the structure.

### BACKGROUND

The present invention relates to reinforced medical implants and methods for making and using the implants.

A variety of skeletal disorders or injuries are benefited by the implant of load-bearing orthopedic devices. For example, spinal discs may be displaced or damaged due to trauma, disease or aging. Disruption of the annulus fibrosus allows the nucleus pulposus to protrude into the vertebral canal, a condition commonly referred to as a herniated or ruptured disc. The extruded nucleus pulposus may press on a spinal nerve, which may result in nerve damage, pain, numbness, muscle weakness and paralysis. Intervertebral discs may also deteriorate due to the normal aging process or disease. As a disc dehydrates and hardens, the disc space height will be reduced leading to instability of the spine, decreased mobility and pain. Sometimes the only relief from the symptoms of these conditions is a discectomy, or surgical removal of a portion or all of an intervertebral disc followed by fusion of the adjacent vertebrae. The removal of the damaged or unhealthy disc will allow the disc space to collapse. Collapse of the disc space can cause instability of the spine, abnormal joint mechanics, premature development of arthritis or nerve damage, in addition to severe pain. Pain relief via discectomy and arthrodesis requires preservation of the disc space and eventual fusion of the affected motion segments.

There have been an extensive number of attempts to develop an acceptable intradiscal implant that could be used to replace a damaged disc and maintain the stability of the disc interspace between the adjacent vertebrae, at least until complete arthrodesis is achieved. The implant must provide temporary support and allow bone ingrowth. Success of the discectomy and fusion procedure requires the development of a contiguous growth of bone to create a solid mass because the implant may not withstand the compressive loads on the spine for the life of the patient.

There is a continuing need for load-bearing medical implants such as orthopedic implants having beneficial strength to resist loads such as compressive, and/or torsional loads. In one particular field of concern, there is a need for spinal interbody fusion implants which have sufficient strength to support the vertebral column until the adjacent vertebrae are fused and which eliminate or at least minimize any permanent foreign body after the fusion.

### SUMMARY

In certain aspects, the present invention provides methods for preparing load-bearing medical implants by hardening a calcium-containing substance in and around one or more organic reinforcing structures, including within an internal porous network of the organic reinforcing structure(s), to form a load-bearing body. Load-bearing implants preparable by such methods are also provided, as are methods of their use. In one embodiment, the invention provides a method for preparing a load-bearing orthopedic or other medical implant. The method includes providing a reinforcing collagen or other polymeric structure, the reinforcing structure including a continuous piece sufficiently rigid to hold shape when positioned within a mold cavity. The reinforcing structure includes a plurality of connected collagen struts defining spaces between the struts, wherein the collagen struts each have an internal interconnected or communicating porous network. The reinforcing structure is placed into a mold cavity; and the mold cavity is filled with a flowable, hardenable calcium-containing material, wherein the filling step is sufficient to drive the flowable, hardenable calcium-containing material into the spaces between the struts and throughout the internal interconnected porous network of the struts. The flowable, hardenable calcium-containing material is then caused to harden to form the load-bearing orthopedic implant.

In another embodiment, the invention provides a load-bearing medical implant comprising a reinforcing collagen structure including a continuous piece having a plurality of connected porous collagen struts defining spaces between the struts, and further wherein said collagen struts each have an internal communicating porous network. The implant further includes a hardened, calcium-containing material formed as a non-sintered load-bearing body encompassing the reinforcing collagen structure. The hardened calcium-containing material fills the spaces between the collagen struts of the reinforcing collagen structure and fills the internal communicating porous network of the collagen struts.

In another embodiment, the invention provides a load-bearing medical implant that comprises a reinforcing structure including a bioresorbable porous polymeric matrix defining an internal interconnected porous network. The implant further comprises a calcium-containing material formed as a hardened, non-sintered load-bearing mass, the load-bearing mass embedding the reinforcing structure and filling the internal communicating porous network of said polymeric matrix.

Additional aspects of the invention relate to methods for treating patients utilizing implant devices as described herein.

These and other aspects of the present invention and related advantages thereof will be apparent from the descriptions herein.

### BRIEF DESCRIPTION OF THE FIGURES

FIG. 1 provides a perspective view of one illustrative medical implant of the invention.

FIG. 2 provides a perspective view of an internal reinforcing structure of the medical implant of FIG. 1.

FIG. 3 illustrates the internal reinforcing structure of FIG. 2 in place within a mold.

FIG. 4 illustrates the arrangement of FIG. 3 after fill of the mold with a calcium-containing implant material.

FIG. 5 is a cross-sectional view of the medical implant depicted in FIG. 1, taken in a plane along line 5-5 of FIG. 1 perpendicular to the longitudinal axis of the implant and viewed in the direction of the arrows.

FIG. 6 is an enlarged view of a designated region of the cross-section shown in FIG. 5 showing an interconnected internal porous network of a portion of a reinforcing structure.

FIG. 7 is an enlarged view of a designated region of the cross-section shown in FIG. 5 showing an alternative interconnected internal porous network of a portion of a reinforcing structure.

FIG. 8 is a perspective view of an illustrative reinforced interbody spinal spacer implant of the invention.

FIG. 9 is a top view of another illustrative reinforced interbody spinal spacer implant of the invention.

FIG. 10 is a cross-sectional view taken in a plane along line 10-10 of FIG. 9 and viewed in the direction of the arrows.

FIG. 11 is a perspective view of another illustrative reinforced interbody spinal spacer implant of the invention.

FIG. 12 is a cross-sectional view taken in a plane along line 12-12 of FIG. 11 and viewed in the direction of the arrows.

### DETAILED DESCRIPTION

For the purposes of promoting an understanding of the principles of the invention, reference will now be made to preferred embodiments and specific language will be used to describe the same. It will nevertheless be understood that no limitation of the scope of the invention is thereby intended, such alterations and further modifications of the invention, and such further applications of the principles of the invention as illustrated herein, being contemplated as would normally occur to one skilled in the art to which the invention relates.

As disclosed above, aspects of the present invention relate generally to reinforced medical implants. One specific aspect of the invention provides load-bearing orthopedic or other medical implants that include a biocompatible, non-sintered load-bearing body formed of a calcium-containing material, wherein the body is reinforced with at least one reinforcing member having an internal interconnected porous network. The load-bearing body of the implant may for example be formed with a flowable calcium phosphate material that is self-hardening. Such a material, in a flowable state, can be passed around and into the pores of the polymeric or other similar reinforcing structure, and then allowed to harden to provide a load-bearing body that partially or completely embeds or encompasses the reinforcing structure.

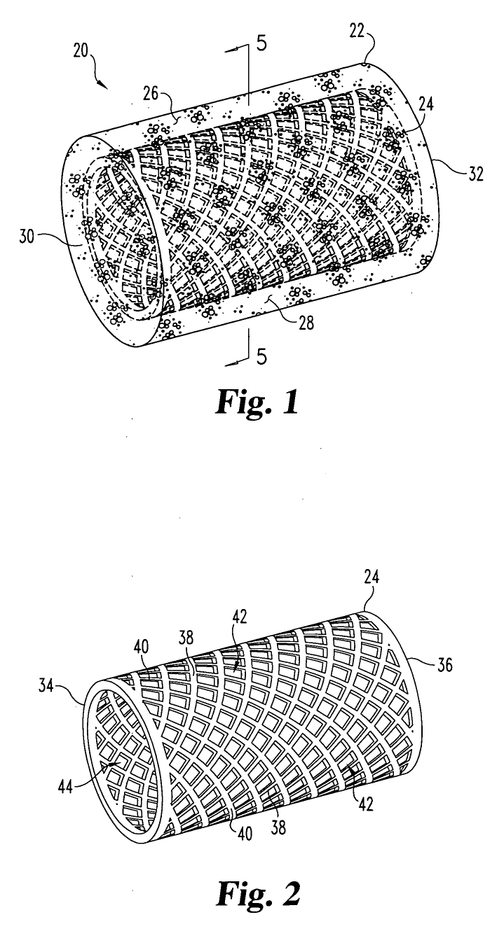

With reference now to FIGS. 1 and 2 together, shown are perspective views of an illustrative reinforced composite implant 20 of the invention and internal reinforcing structure 24 for the implant 20. Implant 20 includes a generally cylindrical implant body 22 having internal reinforcing structure 24 incorporated into body 22. Implant body 22 is made of a non-sintered mass of calcium-containing material. This material can, for example, be provided by a hardenable calcium-containing substance such as a bone cement. Implant body 22 includes a first surface 26 for contacting an adjacent bone surface, e.g. a first vertebral body, and a second surface 28 for contacting a second adjacent bone surface, e.g. a second vertebral body adjacent the first vertebral body. Implant body 22 further includes a first end 30 and a second end 32. As shown in phantom in FIG. 1, internal reinforcing structure 24 in the illustrated embodiment is completely embedded within the body 22 formed of the calcium-containing material.

With reference now particularly to FIG. 2, internal reinforcing structure 24 in the disclosed device 20 is made with a polymeric material. In an especially desired embodiment, reinforcing structure 24 is comprised of collagen. Structure 24 includes a first end 34 and a second end 36 opposite thereto. Extending between ends 34 and 36 is a lattice-work of struts 38 and 40 extending transversely to one another and defining a plurality of openings or spaces 42. Reinforcing structure 24 also defines an internal region 44 which in the illustrated embodiment is a passageway extending between first end 34 and second end 36 and surrounded by the lattice-work of struts 38 and 40.

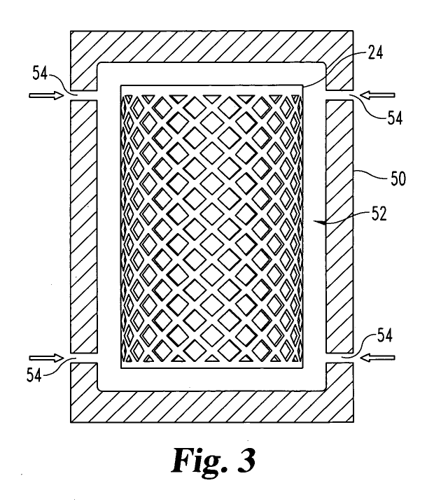

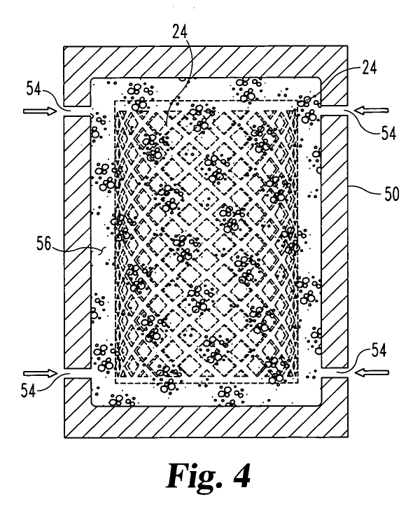

Referring now to FIGS. 3 and 4, an illustrative method for preparing implant device 20 will be discussed. As shown in FIG. 3, reinforcing structure 24 is placed within a mold 50, which can for example be a conventional two or more part mold. Mold 50 defines an internal mold cavity 52 in which reinforcing structure 24 is received. Mold 50 also defines a plurality of openings 54 configured to receive transfer of the calcium-containing material under pressure into the internal mold cavity 52 (see arrows). As shown in FIG. 4, the calcium-containing material, in flowable form, is transferred into the mold cavity 56 sufficiently to fill the cavity. Thus, the finished device in the illustrated method will have surfaces feature corresponding to those defined by the surfaces of the mold cavity 52. The calcium-containing material can then be caused to harden effectively to provide the load-bearing qualities to the finished device. In this regard, the hardening of the calcium-containing material can occur in any suitable manner, including on its own over time at ambient or near ambient temperatures, or can be facilitated by the application of heat or other energy sources and/or pressure.

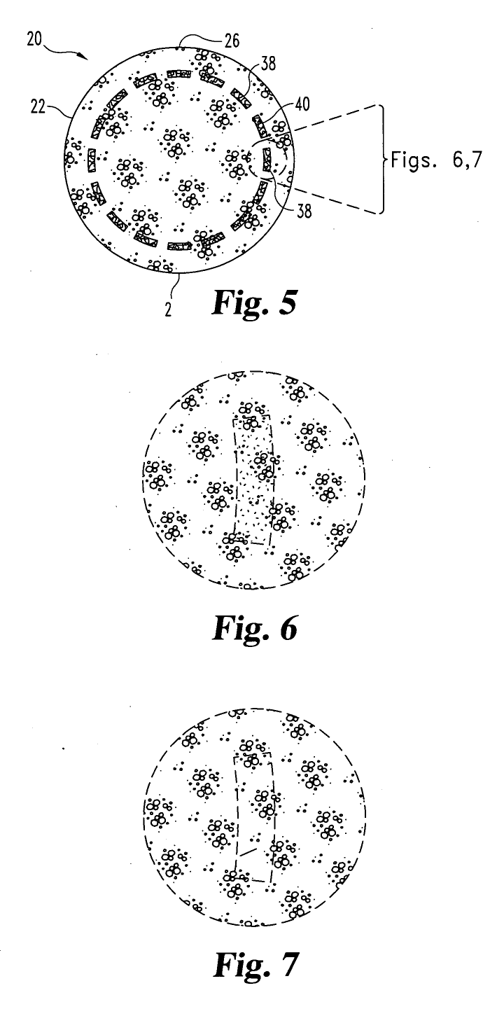

In aspects of the invention, the calcium-containing material is driven into the mold 50 not only sufficiently to fill the mold cavity 52 and to fill the spaces 42 (FIG. 2) of the reinforcing structure 24, but also sufficiently to fill an internal, interconnecting porous network defined in the structural components of reinforcing structure 24 such as struts 38 and 40. In this regard, FIGS. 5-7 illustrate the penetration of the calcium-containing material through such an interconnecting porous structure. Particularly, FIG. 5 provides a cross-sectional view taken in a plane along line 5-5 of FIG. 1 extending perpendicular to the longitudinal axis of device 20. FIGS. 6 and 7 provide enlarged views of the denoted region in FIG. 5 to better illustrate the penetration and filling of the calcium-containing material through an internal interconnected porous network of components of the structure 24. With specific reference to FIG. 6 for purposes of illustration, strut element 38 defines an internal network of pores 58 that are interconnected to one another such that the calcium-containing material can work its way into and through the interconnected porous network and fill the same. Illustrated in FIG. 6 are pores 58 that might be characteristic of a foamed polymeric device, wherein a network of pores includes defined cavities or chambers having curved walls are interconnected by openings between the chambers. Chambers providing pores 58 can, for example, be generally spheroid or ovoid in shape with discontinuities in walls defining them so as to interconnect the pores 58 to one another. Referring now to FIG. 7, another illustrative internal porous network is shown, wherein strut 38 is defined by a fibrous mesh in which a plurality of porous spaces 62 are defined in between intermeshed fibers 60. In both the porous structures illustrated in FIGS. 6 and 7, and in other interconnected porous structures, the interconnection can be of such a nature that the calcium-containing material to be used to prepare the device 20 can be passed under pressure from a first side of the strut 38 or other structural feature and exit a second side thereof. These properties of the interconnected porous network and calcium-containing material will facilitate assuring that substantial penetration of the calcium-containing material into the internal porous network of the reinforcing structure 24 can and will occur in the manufacture of device 20. Accordingly, in the preferred devices as illustrated in FIGS. 6 and 7, the finished, hardened device will include amounts of the calcium-containing material filling the internal porous network of the reinforcing structure 24. Desirably, this penetration and filling is sufficient to provide a substantially continuous phase of the calcium-containing material extending through the struts 38 or other structural elements of reinforcing structure 24. This ensures a highly beneficial, integrated compositing of the polymeric reinforcing structure 24 and the hardened calcium-containing material, to provide improved performance to device 20.

In this regard, the internal polymeric reinforcing structure 24 is desirably of a nature to add to the strength of the device 20 as compared to a corresponding device made without the reinforcing structure. For example, reinforcing structure 24 can add to the tensile and/or shear strength of the device 20. For such purposes, polymeric reinforcing structure 24 is made of a material that is less brittle than that of the hardened calcium-containing material of the device 20. A wide variety of polymeric materials have suitable properties to serve in this capacity.

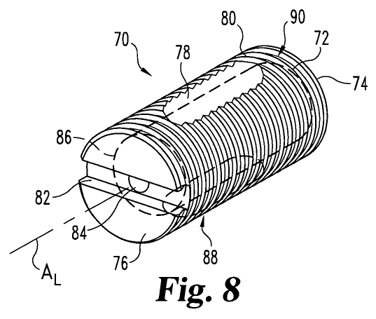

Reinforced devices of the invention can have a wide variety of general shapes and functional implant features, depending upon the intended use of the devices. The general shapes and other surface or functional features of the devices can be imported during a molding process, or provided after a molding process for instance using conventional fabrication techniques such as drilling, milling, grinding, cutting, and the like. FIGS. 8-12 illustrate several advantageous implant devices of the present invention.

In particular, FIG. 8 shows an interbody spinal implant 70 including a load bearing body 72 having disposed therein a polymeric structural reinforcing member 86 incorporated therein as described herein above. Polymeric reinforcing member 86 is generally cylindrical in shape to match the overall shape of the implant 70. Reinforcing member 86 can include a lattice-work as in member 24 (see FIG. 2) or can be a continuous cylindrical plug polymeric reinforcing member having an interconnected internal porous network as discussed herein. Device 70 includes a first end 74 and a second end 76. Device 70 also defines a through-hole 78 that can be filled with an osteogenic material to facilitate spinal fusion, in certain embodiments. Other configurations for holding an osteogenic material will be apparent to those skilled in the art. These include, for example, a hole or indentation that does not extend completely through the load bearing body, but provides a sufficient reservoir for receiving and retaining an osteogenic material. Device 70 also includes an external thread pattern 80 which can be molded into or machined into the device, for example. End 76 of device 70 defines functional features that can serve to engage insertion tools for device 70 and/or to provide a reference that allows a user to determine the position of other structural features on the device 70 by viewing end 76. For example, slot 82 provided in end 76 can serve to engage tools and/or to provide a reference for a user as to the position of through-hole 78. In the illustrated device, slot 82 is positioned generally perpendicularly to through-hole 78. Hole 84 can be provided in a generally central or other suitable location, such as along longitudinal axis A_{L}, and can engage a prong or similar feature of an insertion tool. Spinal spacer device 70 includes a superior vertebral engaging surface 90 and an inferior vertebral engaging surface 88, and is constructed with sufficient compression strength to withstand spinal compression loads without collapse or fracture. Reinforcing structure 86 can provide improved tensile and/or shear strength to contribute to the integrity of the device 70 under non-compressive mechanical loads exerted within the spine including for example bending or torsional loads.

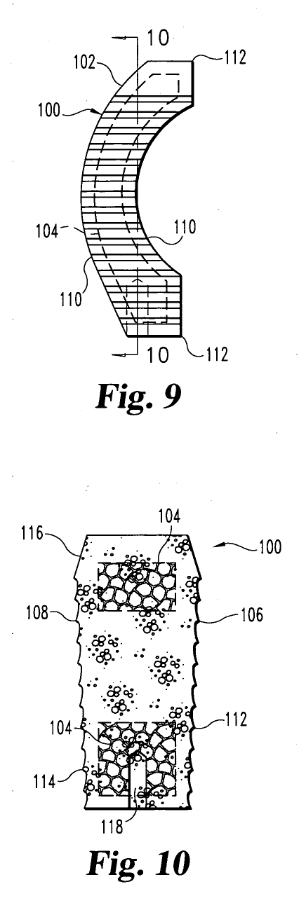

With reference to FIGS. 9 and 10, shown are top and cross-sectional views of another interbody spinal spacer implant device 100 of the invention. Device 100 includes a load bearing body 102 and an internal polymeric reinforcing structure 104. As shown in the cross-section (FIG. 10), polymeric body 104 defines an internal interconnected porous network as discussed herein above, wherein the network is penetrated and filled with the hardened calcium-containing material. Device 100 has a superior surface 106 and an inferior surface 108 that are separated by side walls 110. The load bearing body 102 is shaped substantially like a “C” shape or a crescent shape in the illustrated device 100. The load bearing body 102 can be sized for insertion between two adjacent vertebrae, and in particular for placement within an interbody space between first and second adjacent vertebrae. The superior surface 106 includes surface features 112. Surface features 112 can extend fully across superior surface 106 or in another form surface features 112 can extend partially across superior surface 106. In particular, the surface features 112 are a serrated shape, however other embodiments of the surface features 112 can provide different frictionally-engaging shapes. The inferior surface 108 also includes surface features 114. As shown, surface features 114 are serrations. As with the superior surface 106, surface features 114 can extend partially or fully across inferior surface 108. Surface features 114 can be substantially similar to surface features 112 or can be different therefrom.

As shown in FIGS. 9 and 10, inferior and superior surfaces 106 and 108 each provide a substantially planer overall geometry. The sidewalls 110 are arcuate, providing an overall “C” shape to the spacer body 102. Superior surface 106 and inferior surface 108 can also define an angle or taper. As also illustrated, a tapered portion 116 can be provided at one end of spacer body 102, for instance to provide a leading end for insertion. Load bearing body 102 can also include an instrument hole 118 as best shown in FIG. 10. Instrument hole 118 can be figured to receive and engage a portion of a medical instrument, such as an insertion instrument, to assist a medical practitioner in inserting the load bearing body 102 between adjacent vertebrae. The instrument hole 118 can be various shapes such as, circular, rectangular, or triangular, to name a few, and can include attachment adaptations such as threads if desired.

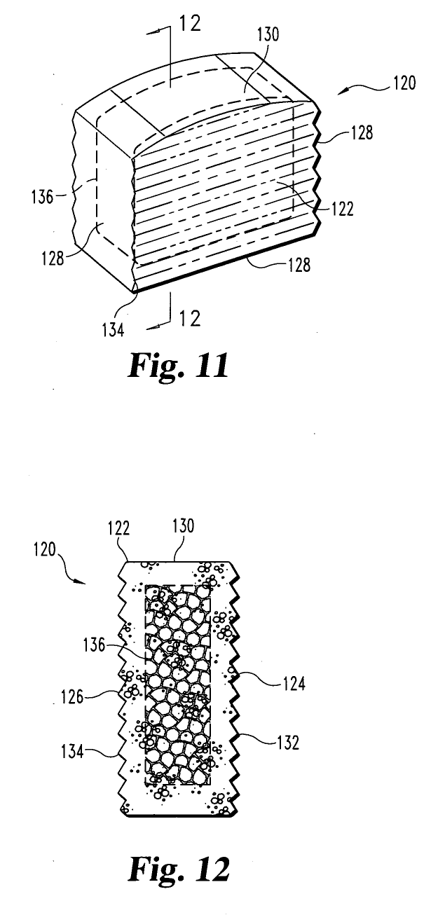

With reference now to FIGS. 11 and 12, shown is another medical implant of the invention. Implant 120 can be used as a load-bearing spinal implant, and in particular aspect as a load-bearing interbody spinal implant. Implant 120 includes a load bearing body 122 made from a calcium-containing material as described herein. Load bearing body 122 includes a superior surface 124 and an inferior surface 126 separating by generally planer sidewalls 128 and arcuate sidewall 130. As shown, superior surface 124 can be generally planer for contacting a surface of a first vertebrae. Similarly, inferior surface 126 can be generally planer for contacting a surface of a second vertebrae. As will be appreciated, in other embodiments, the superior surface 124 and/or inferior surface 126 may be arcuate, or a combination of planer and arcuate, for contacting the surface of the first and/or second vertebrae, respectively. Further, the superior surface 124 can be formed independently of the inferior surface 126. Superior surface 124 includes surface features 132 configured to frictionally engage the first vertebrae. As shown, the surface features 132 are raised portions shaped as serrations. Inferior surface 126 includes surface features 134 configured to engage a second vertebrae. The surface features 134 are also shaped as serrations in the illustrated device. Again, surface features 132 and 134 can be shaped independently of one another in alternative embodiments, and features 132 and 134 can be provided frictionally-engaging shapes other than serrated. As shown, load bearing body 122 is substantially rectangular in shape, with one curved sidewall. In particular, sidewalls 128 are generally planer, whereas sidewall 130 is convexly arcuate, or curved. Curved sidewall 130 can, for example, be configured to correspond to the anterior curvature of adjacent vertebrae between which the load bearing body 122 will be implanted. As shown, implant 120 includes an interior reinforcing structure 136 generally as discussed hereinabove. Thus, a polymeric reinforcing structure 136 can include an internal network of interconnected pores that are filled with the hard calcium-containing material. In the illustrated device 120, internal reinforcing structure 136 has a shape that generally corresponds to that of load bearing body 122, except being smaller in dimension.

Load bearing bodies of implant devices can be formed as non-sintered bodies comprising a calcium-containing implant material. Such bodies are desirably formed with a hardenable calcium-containing material, such as a calcium sulfate or calcium phosphate material. Calcium phosphate materials are preferred.

A wide variety of hardenable calcium-containing materials are known. Calcium phosphate cement (CPC) systems can be used, and typically consist of a powder and a liquid component. The powder component is usually made up of one or more calcium phosphate compounds with or without additional calcium salts. Other additives are usually included in small amounts to adjust the setting times, increase flowability, reduce cohesion or swelling time, and/or introduce macroporosity. Current commercial CPCs include two or more of the following calcium phosphate compounds: amorphous calcium phosphate (ACP), Caₓ(PO₄)_{y}H₂O; monocalcium phosphate monohydrate (MCPH), CaH₄(PO₄)₂H₂O; dicalcium phosphate dihydrate (DCPD), CaHPO₄2H₂O; dicalcium phosphate anhydrous (DCPA), CaHPO₄; precipitated or calcium-deficient apatite (CDA), (Ca,Na)₁₀(PO₄,HPO₄)₆(OH)₂; alpha- or beta-tricalcium phosphate (alpha-TCP, beta-TCP), Ca₃(PO₄)₂; and tetracalcium phosphate (TTCP), Ca₄P₂O₉. Other calcium salts include: calcium carbonate (CC), calcium oxide or calcium hydroxide (CH), calcium sulfate hemihydrate (CSH), and calcium silicate. The liquid component may be one or combinations of the following solutions: saline, deionized H₂O, dilute phosphoric acid, dilute organic acids (acetic, citric, succinic), sodium phosphate (alkaline or neutral), sodium carbonate, sodium alginate, sodium bicarbonate, and/or sodium chondroitin sulfate. The setting reaction product(s) obtained after the cement has set is (are) generally determined by the composition of the powder component and composition and the pH of the liquid component. The setting time (which in certain embodiments can range from about 10 to 60 minutes) is determined for the most part by the composition of the powder and liquid components, the powder-to-liquid ratio (P/L), proportion of the calcium phosphate components (e.g., TTCP/DCPA ratio) and the particle sizes of the powder components. Apatitic calcium phosphate or carbonate-containing apatite (carbonatehydroxyapatite, CHA) with crystallinity (crystal size) similar to that of bone apatite can form before implantation when the cement sets or can result from the in vivo hydrolysis of the non-apatitic setting product (e.g., DCPD) after implantation.

Preferred synthetic calcium phosphate or other materials for use in aspects of the invention are flowable at a low temperature, such as below about 50° C., especially room temperature (about 25° C.), and hardenable at such temperatures. More preferred materials will be flowable at room temperature (about 25° C.) and hardenable at about body temperature (about 37° C.). In certain alternative embodiments, the calcium-containing material can be hardenable upon exposure to pressure and/or a temperature of about 5° C. to about 50° C., typically about 20° C. to 40° C. The calcium:phosphate molar ratio of load bearing bodies formed with calcium phosphate materials can for example be in the range of about 1.3 to 1.7, more typically about 1.5 to 1.7.

Synthetic calcium phosphate materials useful in the devices and methods described herein include but are not limited to those which form a poorly or low crystalline calcium phosphate, such as a low or poorly crystalline apatite, including hydroxyapatite, available from Etex Corporation under the tradename alpha-BSM (and marketed in Europe by Biomet-Merck under the name of BIOBON®). This material is a highly resorbable (complete in vivo resorption in less than a year) cement and includes two powder components: (i) poorly crystalline calcium phosphate (major phase), and (ii) well-crystallized DCPD (Brushite, CaHPO₄.2H₂O). This material has a Ca/P molar ratio less than 1.50. These powders are kneaded with a simple saline solution to form a paste, which upon setting forms a poorly or low crystalline calcium phosphate. For additional information as to these types of materials, reference can be made to U.S. Pat. Nos. 5,783,217; 5,676,976; 5,683,461; 5,650,176; 6,117,456; and PCT International Publication Nos. WO 98/16268, WO 96/39202 and WO 98/16209, all to Lee et al. As defined in the recited patents and herein, by “poorly or low crystalline” calcium phosphate material is meant a material that is amorphous, having little or no long range order and/or a material that is nanocrystalline exhibiting crystalline domains on the order of nanometers or Angstroms.

Many suitable calcium phosphate cements suitable for use in preparing devices described herein comprise tetracalcium phosphate (TTCP) as a main component. For example, U.S. Pat. No. 4,612,053 and EP No. 1172076 disclose cements comprising tetracalcium phosphate and dicalcium phosphate anhydrous (DCPA) as the main components, whereas the U.S. Pat. No. 5,525,148 describes the preparation of a series of calcium phosphate cements which do not contain any TTCP.

Another suitable commercially-available cement is available from Norian Corporation under the tradename Norian SRS. It is a TTCP-containing cement with a secondary component of acidic calcium phosphate, MCPM: Ca(H₂PO₄)2.H2O. This cement has a Ca/P molar ratio slightly greater than 1.50. U.S. Pat. No. 5,152,836 describes a calcium phosphate cement (again with a Ca/P molar ratio slightly greater than 1.50) composed of alpha-TCP (75 wt %), TTCP (18 wt %), DCPD (5 wt %), HA (2 wt %), kneaded into a paste with a relatively concentrated aqueous solution of chondroitin sulphate and sodium succinate. A cement of this type is commercially available under the tradename BIOPEX® (Mitsubishi Material Co.). Another commercially available calcium phosphate cement is known as CALCIBON® (produced and marketed by Biomet-Merck). This material has a Ca/P molar ratio of 1.55, and it includes a mixture of alpha-TCP (58-60 wt %), DCPA.(26-27 wt %), CaCO₃ (12-13 wt %), and HA (2%).

In certain embodiments, a calcium-containing cement used to prepare an implant as described herein can also include a polymer component (e.g. as in the case of polymeric calcium phosphate cements). The polymer component of the cement can for instance include a polyacid such as poly(acrylic acid) or a vinyl compound or derivative thereof such as poly(methyl vinyl ether-maleic acid).

The polymeric reinforcing member or members in implants of the invention can be disposed between the superior surface and the inferior surface of a load-bearing body, and may extend along a length of the load-bearing body, including extending non-parallel, such as obliquely or transverse, or parallel to the superior and inferior surfaces of the body. Additionally, the polymeric reinforcing member(s) may extend non-parallel, including obliquely or transverse, and in other forms may extend parallel, to the central longitudinal axis of the load bearing body of the implants.

Reinforcing structural members may assume a wide variety of shapes. For example, reinforcing structural member may be cylindrical-shaped, spherical, pyramidal, rectangular and other polygonal shapes. FIGS. 1-12 depict a variety of other ways in which the reinforcing structural members may be configured for the implant.

A variety of polymeric materials may be used in the formation of the reinforcing structure. These include as examples natural polymers such as proteins and polypeptides, including fiber-forming proteins such as collagen and elastin. Synthetic polymers may also be employed, including for example biodegradable synthetic polymers such as polylactic acid, polyglycolide, polylactic polyglycolic acid copolymers (“PLGA”), polycaprolactone (“PCL”), poly(dioxanone), poly(trimethylene carbonate) copolymers, polyglyconate, poly(propylene fumarate), poly(ethylene terephthalate), poly(butylene terephthalate), polyethyleneglycol, polycaprolactone copolymers, polyhydroxybutyrate, polyhydroxyvalerate, tyrosine-derived polycarbonates and any random or (multi-)block copolymers, such as bipolymer, terpolymer, quaterpolymer, etc., that can be polymerized from the monomers related to previously-listed homo- and copolymers. It will be well understood that these and other implantable polymeric materials, or combinations thereof, may be used in aspects of the present invention. Biodegradable natural or synthetic polymers are preferred.

Fibrous materials comprising natural polymers, including fibrous protein materials, can be used in the reinforcing structures of the invention. These include, as examples, fibers comprising collagen, elastin, fibronectin, laminin, or other similar structural, fiber-forming proteins. Insoluble, fibrous demineralized bone matrix (DBM) materials can also be used in the invention, alone or in combination with other fibrous materials disclosed herein.

In some forms, the reinforcing structure can be formed with insoluble collagen fibers, soluble collagen, or both. When used together, the soluble collagen and insoluble collagen fibers can first be prepared separately, and then combined. Both the soluble collagen and the insoluble collagen fibers can be derived from bovine hides, but can also be prepared from other collagen sources (e.g. bovine tendon, porcine tissues, recombinant DNA techniques, fermentation, etc.). Suitable collagen materials for use in the invention can be prepared using these or other techniques known in the literature or can be obtained from commercial sources, including for example from Kensey Nash Corporation (Exton, Pa.) which manufactures soluble collagen known as Semed S, fibrous collagen known as Semed F, and a composite collagen known as P1076. Naturally-derived human collagen or recombinant human collagen can also be used as polymeric materials of reinforcing structures of the invention.

The polymeric material is used to form a reinforcing structure having an internal interconnected porous network. Any suitable technique for forming such a structure can be used. For instance, certain polymeric materials may be converted to a flowable liquid material (including, for example, molten states) or the polymer can otherwise be incorporated within or as a flowable liquid material. Such flowable materials can be caused to become a non-flowable solid reinforcing structure having the porous network. Alternatively, monomers for forming the polymeric structure can be caused to polymerize under conditions to form a porous network, e.g. by foaming or other techniques for preparing a polymeric foam or similar porous structure. The reinforcing structure can have a significant level of porosity. For instance, the polymeric structure can have a void volume of at least about 20%, at least about 30%, or at least about 50%. Void volumes in the range of about 20% to about 90% will be typical. In more preferred embodiments described herein, this void volume will be essentially completely (i.e. 97-100%) or at least substantially completely (i.e. 90% or more) occupied by the calcium-containing material forming the load-bearing body.

In certain modes, reinforcing structures can be prepared by forming a suspension of insoluble polymeric solids in a liquid, and then removing the liquid in the formation of the structure, desirably by freezing the liquid and then subliming the resulting frozen solid (e.g. by lyophilization processes). Any suitable liquid can be used for these purposes. The liquid can be an aqueous substance such as water, physiological saline, or phosphate buffered saline. Other liquids can be used alone or in combination with water as well. These may include organic liquids, especially biocompatible organic liquids, for example alcohols such as ethanol. The concentration of solids in the suspension can be used to help to control the ultimate porosity of the polymeric reinforcing portion of the medical implant device, especially in the case of devices prepared by freeze drying. Generally, higher solids concentrations can be useful in the preparation of less porous structures and lower solids concentrations can be useful in the preparation of more porous structures. In certain embodiments herein, the polymeric solids concentration in the suspension will be in the range of about 0.1% to about 10% by weight, and sometimes in the range of about 0.1% to about 5% by weight. It will be understood, however, that other conditions can also be controlled or imparted to vary the porosity of the reinforcing structure. For instance, the dispersion pH, lyophilization cycle, and other factors can be varied to vary the porosity of the solid structure once dried. In addition, the solid structure can be subjected to further processing to affect its porosity, the further processing including for example re-wetting, compression and crosslinking so as to reduce the porosity and increase the density of the structure.

The strength of the polymeric reinforcing structure, especially but not solely in the case of structures formed from collagen or other natural polymeric fibers, can be enhanced by crosslinking the structure. Suitable crosslinking techniques include for example chemical reaction, the application of energy such as radiant energy (e.g. UV light or microwave energy), drying and/or heating and dye-mediated photo-oxidation; dehydrothermal treatment; enzymatic treatment; and others. Chemical crosslinking agents are used in certain embodiments, including those that contain bifunctional or multifunctional reactive groups, and which react with collagen. Chemical crosslinking can be introduced by exposing the collagen-mineral composition to a chemical crosslinking agent, either by contacting with a solution of the chemical crosslinking agent or by exposure to the vapors of the chemical crosslinking agent. This contacting or exposure can occur before, during or after a molding operation to form the polymeric reinforcing structure. In any event, the resulting material can then be washed to remove substantially all remaining amounts of the chemical crosslinker if needed or desired for the performance or acceptability of the final bone implant. Suitable chemical crosslinking agents include mono- and dialdehydes, including glutaraldehyde and formaldehyde; polyepoxy compounds such as glycerol polyglycidyl ethers, polyethylene glycol diglycidyl ethers and other polyepoxy and diepoxy glycidyl ethers; tanning agents including polyvalent metallic oxides such as titanium dioxide, chromium dioxide, aluminum dioxide, zirconium salt, as well as organic tannins and other phenolic oxides derived from plants; chemicals for esterification or carboxyl groups followed by reaction with hydrazide to form activated acyl azide functionalities; dicyclohexyl carbodiimide and its derivatives as well as other heterobifunctional crosslinking agents; hexamethylene diisocyante; sugars, including glucose, are also useful for introducing crosslinking.

In certain embodiments, reinforcing structures used in preparing medical implant devices of the invention can exhibit sufficient rigidity to be self-supporting, and/or to withstand the forcible infiltration of the flowable calcium-containing material without significant permanent deformation. Crosslinking or other preparative techniques can be used to impart such physical properties to the reinforcing structure. In some variants, the reinforcing structure can be deformable but resilient, exhibiting shape memory. For example, a resilient reinforcing structure may undergo some deformation during the process of forming the implant device, but exhibit the capacity to return to or toward its original shape prior to hardening of the calcium-containing material.

The interconnected pores of the porous network of the reinforcing structure desirably include an interconnected macroporous network, wherein the size of the macropores is at least about 50 microns, typically in the range of about 100 to 1000 microns, and more typically in the range of about 100 to 500 microns. The particle size of the solids of the calcium-containing material will be sufficiently small to infiltrate the internal porous network to fill the same in certain methods of preparing inventive implants. In this regard, desired such particle sizes will be less than about 50 microns, more typically less than about 40 microns. Small particle sizes of less than about 30 microns can be beneficially used.

The overall reinforced implant device, once formed, can exhibit porosity. In certain desirable forms, the device will exhibit only microporosity, with only pores of a size less than about 10 microns being present, and in some forms less than about 5 microns. Such small pores in the device can contribute to a relatively highly dense, strong load-bearing body for the device. Overall, the porosity of the device can in certain embodiments be about 20% to about 70%, more typically in the range of about 30% to about 60%. It will be understood, however, that higher or lower porosities (including essentially non-porous bodies) can also be manufactured in accordance with aspects of the present invention.

The overall reinforced implant device can exhibit significant load-bearing capacity. In more desirable embodiments, the wet compression strength of the implant device will be at least about 10 Mega Pascals (MPa), and in certain embodiments within the range of about 10 MPa to about 100 MPa. Higher strength implants, for instance having a wet compression strength of at least about 40 MPa, e.g. about 40 MPa to about 200 MPa, can be used to advantage in medical applications in which the implants will be subjected to more severe compressive loads. It will be understood that compression strengths higher or lower than these values may also be exhibited in certain implants of the invention.

In advantageous embodiments, a calcium-containing material forming a non-sintered load-bearing body can incorporate an osteogenic substance, such as an osteogenic protein. In certain forms, the osteogenic substance can be admixed with the calcium-containing material prior to forming the load-bearing body and while the material remains in a flowable state. The flowable, calcium-containing osteogenic material can then be used to form the load-bearing body around the reinforcing structure. On other forms, the osteogenic substance can be applied onto and/or into the load-bearing body after the body is formed. This can be achieved, for instance, by contacting the formed body with the osteogenic substance, preferably with the substance in a liquid carrier such as an aqueous solution or other suitable medium. Liquid preparations containing the osteogenic substance can be contacted with the formed body in any suitable fashion, including for instance immersion of the body in the liquid preparation, spraying the body with the liquid preparation, dripping or pouring the preparation onto the body, and the like. The contacting can be conducted to incorporate the osteogenic substance in the body in any of a variety of configurations, including as examples surface coating the body with the osteogenic substance, regionally incorporating the ostegenic substance in the body, or incorporating the osteogenic substance in a substantially homogenous distribution throughout the body. These and other manners in which the osteogenic substance can be beneficially combined with the load-bearing body will be apparent to those skilled in the field from the descriptions herein.

A variety of osteogenic substances can be used in or in conjunction with devices and methods of the invention. Suitable osteogenic materials can include a growth factor that is effective in inducing formation of bone. Desirably, the growth factor will be from a class of proteins known generally as bone morphogenic proteins (BMPs), and can in certain embodiments be recombinant human (rh) BMPs. These BMP proteins, which are known to have osteogenic, chondrogenic and other growth and differentiation activities, include rhBMP-2, rhBMP-3, rhBMP4 (also referred to as rhBMP-2B), rhBMP-5, rhBMP-6, rhBMP-7 (rhOP-1), rhBMP-8, rhBMP-9, rhBMP-12, rhBMP-13, rhBMP-15, rhBMP-16, rhBMP-17, rhBMP-18, rhGDF-1, rhGDF-3, rhGDF-5, rhGDF-6, rhGDF-7, rhGDF-8, rhGDF-9, rhGDF-10, rhGDF-11, rhGDF-12, rhGDF-14. For example, BMP-2, BMP-3, BMP-4, BMP-5, BMP-6 and BMP-7, disclosed in U.S. Pat. Nos. 5,108,922; 5,013,649; 5,116,738; 5,106,748; 5,187,076; and 5,141,905; BMP-8, disclosed in PCT publication WO91/18098; and BMP-9, disclosed in PCT publication WO93/00432, BMP-10, disclosed in U.S. Pat. No. 5,637,480; BMP-1, disclosed in U.S. Pat. No. 5,639,638, or BMP-12 or BMP-13, disclosed in U.S. Pat. No. 5,658,882, BMP-15, disclosed U.S. Pat. No. 5,635,372 and BMP-16, disclosed in U.S. Pat. Nos. 5,965,403 and 6,331,612. Other compositions which may also be useful include Vgr-2, and any of the growth and differentiation factors [GDFs], including those described in PCT applications WO94/15965; WO94/15949; WO95/01801; WO95/01802; WO94/21681; WO94/15966; WO95/10539; WO96/01845; WO96/02559 and others. Also useful in the present invention may be BIP, disclosed in WO94/01557; HP00269, disclosed in JP Publication number: 7-250688; and MP52, disclosed in PCT application WO93/16099. The disclosures of all of these Patents and applications are hereby incorporated herein by reference. Also useful in the present invention are heterodimers of the above and modified proteins or partial deletion products thereof. These proteins can be used individually or in mixtures of two or more. rhBMP-2 is preferred.

The BMP may be recombinantly produced, or purified from a protein composition. The BMP may be homodimeric, or may be heterodimeric with other BMPs (e.g., a heterodimer composed of one monomer each of BMP-2 and BMP-6) or with other members of the TGF-beta superfamily, such as activins, inhibins and TGF-beta 1 (e.g., a heterodimer composed of one monomer each of a BMP and a related member of the TGF-beta superfamily). Examples of such heterodimeric proteins are described for example in Published PCT Patent Application WO 93/09229, the specification of which is hereby incorporated herein by reference. The amount of osteogenic protein useful herein is that amount effective to stimulate increased osteogenic activity of infiltrating progenitor cells, and will depend upon several factors including for instance the particular protein being employed.

Other therapeutic growth factors or substances may also be used in implant devices of the present invention. Such proteins are known and include, for example, platelet-derived growth factors, insulin-like growth factors, cartilage-derived morphogenic proteins, growth differentiation factors such as growth differentiation factor 5 (GDF-5), and transforming growth factors, including TGF-α and TGF-β.

The osteogenic proteins or other biologically active agents, when used in the present invention, can be provided in liquid formulations, for example buffered aqueous formulations. In certain embodiments, such liquid formulations can be mixed with, received upon and/or within, or otherwise combined with a formed implant device body as discussed above, or can be admixed or otherwise combined with materials ultimately used for form the device body. One suitable rhBMP-2 formulation is available from Medtronic Sofamor Danek, Memphis, Tenn., with its INFUSE® Bone Graft product. In certain embodiments, a liquid recombinant BMP-2 preparation having a concentration of about 0.4 mg/ml to about 4 mg/ml is used in the preparation of the formed implant device and/or combined with a carrier material to be used in conjunction with a formed implant device of the invention.

Other additives may be included in the calcium-containing compositions that form the load-bearing bodies of the present invention to adjust their properties, including supporting or strengthening filler materials and/or pore forming agents. Illustrative porosifying agents include ice, biodegradable polymers (e.g. such as those discussed above) and salts, wherein these porosifying materials melt, dissolve or degrade more rapidly than the calcium-containing phase incorporating them. Illustrative strengthening filler materials include reinforcing fibers, which can be made of a suitable polymeric material. The reinforcing fibers may be made of any suitable biodegradable material by methods known to the art or may be commercially available fibers. Polyglycolide (PGA) fibers are available from several commercial sources including Albany International, Sherwood Davis & Geck and Genzyme Surgical Products. Reinforcing fibers are preferably synthetic fibers, and are preferably of a length short enough not to interfere with processability of the calcium-containing material, e.g., less than about 1 cm. The reinforcing fibers will typically have a relatively small diameter, for instance between about 5 microns and about 50 microns.

As mentioned above, any thru-holes or other apertures or discontinuities in the medical implant body may be filled with an osteogenic material. Any suitable osteogenic material or composition is contemplated, including autograft, allograft, xenograft, demineralized bone, synthetic and natural bone graft substitutes, such as bioceramics, polymers, and osteoinductive factors. The terms osteogenic material or osteogenic composition as used herein mean virtually any material that promotes bone growth or healing including autograft, allograft, xenograft, bone graft substitutes and natural, synthetic and recombinant proteins, nucleotide sequences (e.g. genes such as growth factor genes), hormones and the like.

Autograft can be harvested from locations such as the iliac crest using drills, gouges, curettes, trephines and other tools and methods which are well known to surgeons in this field. Preferably, autograft is harvested from the iliac crest with minimally invasive surgery. The osteogenic material may also include bone reamed away by the surgeon while preparing the end plates for the implant.

Advantageously, where autograft is chosen as the osteogenic material, only a very small amount of bone material is needed to pack the thru-hole, if present. The autograft itself is not required to provide structural support as this is provided by the implant. The donor surgery for such a small amount of bone is less invasive and better tolerated by the patient. There is usually little need for muscle dissection in obtaining such small amounts of bone. The present invention therefore eliminates or minimizes many of the disadvantages of employing autograft.

When an osteogenic protein is used in a separate composition to be combined with the formed implant device, or implanted along with the device, it can be combined with an appropriate carrier material. Potential carriers include calcium sulphates, polylactic acids, polyanhydrides, collagen, calcium phosphates, hyaluronic acid, polymeric acrylic esters and demineralized bone, as examples. The carrier may be any suitable carrier capable of delivering the protein. Desirably, the carrier is capable of being eventually resorbed into the body. One desirable carrier is an absorbable collagen sponge marketed by Integra LifeSciences Corporation under the trade name Helistat® Absorbable Collagen Hemostatic Agent. Another good carrier is a biphasic calcium phosphate ceramic. Ceramic blocks and granules are commercially available from Sofamor Danek Group, B. P. 4-62180 Rang-du-Fliers, France and Bioland, 132 Rou d Espangne, 31100 Toulouse, France. The osteognic protein can be introduced into the carrier in any suitable manner. For example, the carrier may be soaked with a solution containing the osteogenic protein.

Implant devices of the invention can be prepared to have minimal or no metallic components. Such implants will be advantageous, for example, in minimizing the metal artifact in computer tomography (CT) or magnetic resonance imaging (MRI) which makes post-operative complications diagnosis easier. It will also be easier to assess the fusion radiographically using such implants. Moreover, the calcium-containing materials forming the body of the implant devices may degrade over time and be replaced by bone. Additionally, direct bone apposition to the calcium-containing material instead of possible fibrous tissue interfaces with metal devices will be advantageous.

In yet other aspects of the present invention, methods for treating patients using implant devices of the invention are provided, desirably by implanting such devices at a site at which bone growth is desired. Preferred such methods are conducted to promote fusion bone growth between adjacent vertebrae. In one form of the invention, a method includes providing a first interbody fusion implant described herein, such as one having a load bearing body with a reinforcing structure disposed therein. The implant selected is of the appropriate dimensions, based on the size of the cavity created and the needs of the particular patient undergoing the fusion. The adjacent vertebrae are prepared to receive the spacer in an intervertebral space between adjacent vertebrae according to conventional procedures. The spacer is mounted on an instrument known to the art, preferably via an instrument attachment hole. An osteogenic material may optionally be placed within a thru-hole, or gap, of the implant should one be present. The implant is then inserted into the cavity created between the adjacent vertebrae to be fused. Once the implant is properly oriented within the intervertebral space, the implant may be disengaged from the instrument. In certain forms of the invention, a second implant is inserted into the intervertebral space after the first implant is properly positioned, resulting in bilateral placement of the spacers. Osteogenic material may also optionally be placed within those implants having thru-holes, and/or in and around the implants positioned within the intervertebral space.

The uses of the terms “a” and “an” and “the” and similar references in the context of describing aspects of the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected. In addition, all references cited herein are indicative of the level of skill in the art and are hereby incorporated by reference in their entirety.

What is claimed is:

1. A method for preparing a load-bearing medical implant, comprising:
providing a reinforcing collagen structure, said reinforcing collagen structure including a continuous piece sufficiently rigid to hold shape when positioned within a mold cavity, said reinforcing collagen structure including a plurality of connected collagen struts defining spaces between the struts, and further wherein said collagen struts each have an internal communicating porous network;
inserting the reinforcing collagen structure into a mold cavity; and
filling the mold cavity having the reinforcing collagen structure inserted therein with a flowable, hardenable calcium-containing material, said filling sufficient to drive the flowable, hardenable calcium-containing material into the spaces between the collagen struts and throughout the internal interconnected porous network of the collagen struts; and
causing the flowable, hardenable calcium-containing material to harden to form the load-bearing orthopedic implant.

2. The method of claim 1, wherein said calcium-containing material comprises a calcium phosphate cement.

3. The method of claim 1, wherein said load-bearing orthopedic implant is an interbody spinal fusion implant sized for receipt in an interbody space between adjacent vertebral bodies.

4. The method of claim 1, wherein said reinforcing collagen structure comprises chemically-crosslinked collagen.

5. The method of claim 1, wherein said hardened calcium-containing material is bioresorbable.

6. The method of claim 1, wherein said load-bearing orthopedic implant exhibits a compression strength of at least 10 MPa.

7. The method of claim 1, wherein said collagen struts exhibit a void volume of at least about 50%, and wherein said void volume is essentially completely occupied by the calcium-containing material.

8. A load-bearing medical implant, comprising:
a reinforcing collagen structure, said reinforcing collagen structure including a continuous piece having a plurality of connected porous collagen struts defining spaces between the struts, and further wherein said collagen struts each have an internal interconnected porous network;
a hardened, calcium-containing material formed as a non-sintered load-bearing body encompassing said reinforcing collagen structure, said hardened calcium-containing material filling the spaces between the collagen struts of the reinforcing collagen structure and filling the internal interconnected porous network of said collagen struts.

9. The implant of claim 8, wherein said calcium-containing material comprises calcium phosphate cement.

10. The implant of claim 8, which is an interbody spinal fusion implant sized for receipt in an interbody space between adjacent vertebral bodies.

11. The implant of claim 8, wherein said reinforcing collagen structure comprises chemically-crosslinked collagen.

12. The implant of claim 8, wherein said calcium-containing material is bioresorbable.

13. The implant of claim 8, which exhibits a compressive strength of at least 10 MPa.

14. The implant of claim 8, wherein said porous collagen struts exhibit a void volume of at least about 50%, and wherein said void volume is essentially completely occupied by the calcium-containing material.

15. The implant of claim 14, wherein the calcium-containing material comprises a calcium phosphate cement, and wherein the load-bearing implant exhibits a compressive strength of at least about 10 MPa.

16. The implant of claim 8, wherein said calcium-containing material carries an osteoinductive protein.

17. The implant of claim 16, wherein said osteoinductive protein is a bone morphogenic protein.

18. A load-bearing medical implant, comprising:
(i) a reinforcing structure comprising a bioresorbable porous polymeric matrix defining an internal interconnected porous network; and
(ii) a calcium-containing material formed as a hardened, non-sintered load-bearing mass, said mass embedding said reinforcing structure and filling the internal interconnected porous network of said polymeric matrix.

19. The implant of claim 18, wherein said calcium containing material comprises an osteogenic protein.

20. The implant of claim 19, wherein said osteogenic protein is a recombinant human bone morphogenic protein.

21. The implant of claim 18, wherein said polymeric matrix comprises collagen.

22. The implant of claim 18, wherein said porous polymeric matrix exhibits a void volume of at least 50%, with said void volume occupied by said calcium-containing material.

23. A method for treating a patient, comprising implanting in the patient an implant according to claim 8.

24. A method for treating a patient, comprising implanting in the patient an implant according to claim 18.

25. A method for preparing a load-bearing medical implant, comprising:
(i) providing a reinforcing structure comprising a bioresorbable porous polymeric matrix defining an internal interconnected porous network;
(ii) forming a flowable, hardenable calcium-containing material into a mass embedding said reinforcing structure and filling the internal interconnected porous network of said polymeric matrix; and
(iii) causing said flowable, hardenable calcium-containing material to harden.
